(19) United States
(12) Patent Application Publication
(10) Pub. No.: US 2011/0251489 A1
(43) Pub. Date: Oct. 13, 2011

(54) ULTRASOUND MONITORING SYSTEMS, METHODS AND COMPONENTS

(75) Inventors: Jimin ZHANG, Bellevue, WA (US); Randy SERROELS, Sammamish, WA (US); Ingrid LIN, Seattle, WA (US); Robert Bruce HUBLER, Woodinville, WA (US); Joseph Patrick SULLIVAN, Issaquah, WA (US); Paul C. LEONARD, Woodinville, WA (US); Joel ARAGON, Snohomish, WA (US); Luke FRYER, Seattle, WA (US); Harold A. BROWN, Seattle, WA (US); Clare LONG, Edmonds, WA (US); Nathan J. DALE, Bothell, WA (US)

(73) Assignee: PHYSIOSONICS, INC., Bellevue, WA (US)

(21) Appl. No.: 12/756,108

(22) Filed: Apr. 7, 2010

(51) Int. Cl. A61B 8/00

(52) U.S. Cl. 600/459

(57) ABSTRACT

Ultrasound monitoring systems and components used in ultrasound monitoring systems, such as Transcranial Dopper (TCD) systems, are disclosed. Components include framework systems for mounting, locating and maintaining one or more ultrasound probes in contact with an anatomical surface, adjustable probe mounting systems, and probe interface components providing an acoustically transmissive interface between a probe mounting system and the emissive face of the ultrasound probe.

### TECHNICAL FIELD OF THE INVENTION

The present invention relates to ultrasound monitoring systems and components used in ultrasound protocols and monitoring systems, such as transcranial Doppler (TCD) systems, including framework systems for mounting, locating and maintaining one or more ultrasound transducer(s), or probe(s), in contact with an anatomical surface (e.g., skin, skull) of a subject, adjustable probe mounting systems, and probe interface components providing an interface between an ultrasound probe mounting system and the probe and, optionally, providing an acoustically transmissive coupling for contacting a subject's skin or another anatomical surface. Methods for using the probe mounting systems, interface components and/or framework structure, and for adjusting the acoustic illumination area of ultrasound probes with respect to a target site are also disclosed.

### BACKGROUND OF THE INVENTION

In the field of medical imaging, ultrasound systems may be used in various modes to produce images of objects or structures within a patient. In a transmission mode, an ultrasound transmitter is placed on one side of an object (e.g., a body portion) and ultrasound beams are transmitted into the object (e.g., body portion, tissue, etc.) and ultrasound receive beams are acquired by an ultrasound receiver. An image may be produced in which the brightness of each image pixel is a function of the amplitude of the ultrasound that reaches the receiver (attenuation mode), or the brightness of each pixel may be a function of the time required for the sound to reach the receiver (time-of-flight mode). Alternatively, if the receiver is positioned on the same side of the object as the transmitter, an image may be produced in which the pixel brightness is a function of the amplitude of reflected ultrasound (reflection or backscatter or echo mode). In a Doppler mode of operation, the tissue (or object) is imaged by measuring the phase shift of the ultrasound wave reflected from the tissue (or object) back to the receiver.

When used for imaging, ultrasound probes are provided with several piezoelectric elements arranged in an array and driven by different voltages. By controlling the phase and amplitude of the applied voltages, ultrasound waves combine to produce a net ultrasound wave that travels along a desired beam direction and may be focused at a selected point along the beam. By controlling the phase and the amplitude of the applied voltages, a focal point or area of beams can be moved in a plane to scan a target area. Many types of ultrasound imaging systems, transducers and probes are well known in the art.

Doppler ultrasound techniques, as mentioned, measure the phase shift (the “Doppler Effect”) of reflected sound, which indicates the velocity of the reflecting material. Long-standing applications of Doppler ultrasound include monitoring of the fetal heart rate during labor and delivery and evaluating blood flow in the carotid artery. Transcranial Doppler (TCD) ultrasound technology provides detection and measurement of blood flow in a variety of intracranial arteries by applying ultrasound to areas or windows of the skull where the bone is relatively thin. The frequency of the Doppler signal is adjusted and transmitted in a pulsed wave rather than continuous wave mode to augment the transmission of ultrasound waves through the skull. Blood flow velocities from the cerebral arteries, the internal carotids, the basilar and the vertebral arteries can be sampled by altering the probe location and angle, and the instrument's depth setting. The most common windows in the cranium are located in the orbit (of the eye), and in the temporal and suboccipital regions.

TCD ultrasonography provides an easy-to-use, non-invasive, non-radioactive, and relatively inexpensive method to assess intracerebral hemodynamics with temporal resolution and provides reliable detection of cerebral perfusion changes. Using TCD ultrasonography, cerebrovascular responsiveness to various physiological and pharmacological challenges can be assessed instantaneously, and various cerebral circulatory tests can be repeated often and safely. Rapid changes of cerebral perfusion over time can be easily followed, documented and analyzed. The use of Doppler ultrasound has expanded greatly in the past two decades, and Doppler ultrasound is now used in many medical specialties, including cardiology, neurology, radiology, obstetrics, pediatrics, and surgery.

In operation, a TCD acoustic source/detector combination, such as an ultrasound source/detector probe, is contacted to and held against a patient's skin, for example at a temporal window, and manipulated by a trained sonographer to find blood vessels of interest. An acoustically transmissive path is generally provided between the emissive face of the transducer and the skin surface using a gel material having high acoustic transmissivity. The sonographer is generally required to monitor and adjust the position of the ultrasound source/detector probe during an examination to maintain focus on the blood vessel(s) of interest as the patient breathes and moves. For longer term monitoring applications, an ultrasound source/detector probe may be stably mounted, or held, in proximity to a patient's body surface. For central nervous system (CNS) target sites, the acoustic source/detector probe is stably mounted, or held, in proximity to a cranial window and manipulated until a desired target site, such as a cranial blood vessel, is located. The acoustic source/detector probe combination is preferably provided as a unitary component, but separate acoustic source and detector components may also be used.

Various types of acoustic transducers and acoustic transducer arrays may be used as acoustic source/detector probe assemblies and acoustic data acquisition components. A single acoustic transducer, or a singer acoustic transducer array may be operated both as a source and a detector, or separate source and detector transducers or transducer arrays may be provided as ultrasound probes. Conventional PZT acoustic transducers may be implemented as acoustic data acquisition components. Acoustic transducer arrays comprising cMUT and PVDF cells or elements may also be used. PZT, cMUT and PVDF acoustic transducers and arrays may be combined in various data acquisition components and operated in acoustic source and/or receiver modes. Various types of acoustic transducer combinations and arrays are described in U.S. Pat. No. 7,547,283, the disclosure of which is incorporated by reference herein in its entirety.

One drawback of measuring physiological parameters using a standard TCD probe is that identifying a desired target site using a TCD probe is challenging and generally requires a trained, experienced sonographer to find and (acoustically) illuminate a desired target site, such as the middle cerebral artery (MCA). When longer term monitoring of physiological parameters using a TCD probe is required, a cumbersome and generally uncomfortable headset having the TCD probe mounted on it is generally mounted on the subject's head to stabilize the transducer position and reduce the effects of patient movement and other disturbances on the position of the probe. The sonographer may be required to monitor acoustic readings and reposition the transducer intermittently to maintain the focus on the desired data acquisition area.

U.S. Pat. No. 6,682,483 discloses the use of a low-profile, easily attached transducer pad that may be mounted directly on a patient's skull to provide long-term unattended Doppler ultrasound monitoring in spite of motion of the patient or the pad. The low-profile transducer probe may be adhered, lightly taped, strapped, banded or otherwise easily attached to the portion of the body where the vascular diagnosis or monitoring is required and used to track and maintain focus on multiple desired blood vessels.

U.S. Pat. No. 7,547,283 discloses a head-set arrangement wherein a transducer array and array electronics are permanently mounted on a structure facilitating communication to and from a controller component. An acoustic transmission component may be provided as a single use component and may be affixed to an exposed surface of the transducer array prior to mounting on a subject's body surface. Various combinations of single use components and elements are described.

Long-term ambulatory TCD monitoring using a transducer probe having a lightweight protective cover that mounts on the stem of eyeglasses is described in Long-Term Ambulatory Monitoring for Cerebral Emboli Using Transcranial Doppler Ultrasound, Mackinnon et al., Stroke 2004; 35; 73-38; originally published online Dec. 18, 2003. The ambulatory TCD system included a small, lightweight battery-powered Doppler unit with flash storage capacity communicating with the transducer probe that could be carried in a pocket.

U.S. Pat. No. 5,514,146 discloses various adjustable support mechanisms for adjusting at least one sonographic probe and fixing it on the skull of a patient. Several headframe probe holders for use in TCD examinations and protocols are available commercially, providing various configurations and levels of adjustability of the headframe as well as the position of the probe(s).

The disclosure provided herein is directed to ultrasound monitoring systems, methods and components for use in monitoring physiological conditions and parameters accurately and without requiring frequent intervention of a trained sonographer.

### SUMMARY

In one aspect, ultrasound monitoring systems of the present invention comprise one or more ultrasound transducer(s), or ultrasound probe(s), that communicate with one or more controller(s) (via wired and/or wireless communication protocols and power transfer mechanisms) that operate the probe(s) and acquire, process, analyze and/or display data. The ultrasound monitoring systems and components of the present invention are particularly suitable for use with transcranial Doppler (TCD) systems, although they may be adapted for use with other types of ultrasound protocols and monitoring systems. Additional components and features that facilitate the use, positioning and operation of ultrasound probe(s) to acquire data, such as frame members for mounting on a patient to position probe(s), adjustable probe mounts, probe interface components, and the like, are also disclosed. Many or all of these components may be provided as single use or individual-specific or probe-specific or protocol-specific components.

Specialized framework components may be provided for mounting to and stable positioning on different portions of a subject's anatomy and are designed with one or more integral or detachable probe mount(s) for receiving an ultrasound transducer housing, or probe, and positioning the probe in proximity to an anatomical surface of a subject, such as a skin surface. Bands or similar components may be provided to at least partially underlie the framework component, providing a comfortable interface with a subject's anatomical surface and providing an effective mounting surface for a framework component. In one embodiment, a band may be provided as a flexible, elastic component sized and configured to contact (directly or indirectly) a desired location on a subject's anatomy and provide a contact surface for a framework component. In some embodiments, bands provided for contacting a subject are adjustable and may incorporate padding or comprise a material that's comfortable against a skin surface. In some embodiments, bands provided for contacting a subject and providing an interface for positioning the framework component may comprise both flexible and substantially rigid portions. In some embodiments, such bands may be provided with stiff framework interface member(s) that mate with a corresponding interface member(s) provided on the framework component for stably and positively positioning the framework component on the band.

An ultrasound probe mount may be provided as part of the framework component or may be provided as a separate component mountable to the framework component and is configured to receive an ultrasound probe. The ultrasound probe mount is generally adjustable with respect to the framework component and a subject's anatomical surface in at least two dimensions to provide convenient and stable positioning of an ultrasound emitting face of an ultrasound probe at desired anatomical locations on a subject. In some embodiments, the ultrasound probe mount may be adjustable along at least three adjustment paths. In some embodiments, the probe mount is adjustable along at least two linear paths and at least one rotational path. In some embodiments, the probe mount has at least one curved, at least partially spherical surface adapted to contact a curved surface of a probe housing or intermediate structure, providing for adjustment of the probe with respect to the probe housing (and subject) with multiple degrees of freedom by interaction of the curved surfaces. In some embodiments, a gimbal-like mechanism may be provided for adjustment of an ultrasound probe in a probe mount. In yet other embodiments, the probe mount is adjustable along a z-axis, toward and away from an anatomical surface of a subject. In still other embodiments, the probe mount may be adjustable along at least one adjustment path in each of three dimensions. In many embodiments, the ultrasound probe mount and/or ultrasound probe are lockable in a desired adjustment position following adjustment of the ultrasound probe and probe mount.

A probe interface component is generally provided integrally with or mountable in or on the ultrasound probe housing and comprises an acoustically transmissive material providing generally high fidelity acoustic transmission between an emissive transducer face of the ultrasound probe and a subject's anatomical surface. In some embodiments, the probe interface component may be integrated with the probe mount, providing an integrated, multifunctional component for receiving an ultrasound probe and mounting the probe, along with the integrated interface and probe mount, on a framework structure positioned on a subject's anatomical surface. In other embodiments, the probe interface component and the probe mount may be provided as separate, mating components that may be combined to provide a stable combination and are also detachable from one another. Specialized framework components, probe mounts, and/or probe interface components may be provided as subject-specific, protocol-specific and/or probe-specific components. These components may be designed and configured as single use or multiple use components.

Probe mount and interface components may be sized and configured to match a variety of ultrasound transducers and probes used with a variety of ultrasound diagnostic systems, monitoring systems, imaging systems, and the like. In one embodiment, an ultrasound probe may be coupled to a single use probe interface component, and that probe assembly may be inserted into an adjustable probe mount provided separately from and mountable on a frame component. An adjustable probe mount may alternatively be provided as part of a frame component. When the framework structure is mounted on a subject's anatomical surface, an emissive face of the ultrasound probe(s) is exposed through a port in the probe mount and positioned in proximity to the subject's anatomical surface, such as a skull surface. The emissive face of the probe generally contacts a probe interface component having an acoustically transmissive member that provides a high fidelity acoustic path between the emissive face of the probe and the subject's surface. In some embodiments, an acoustically transmissive material, such as an acoustic gel, may be applied to the emissive face of the probe, and the probe may then be positioned in proximity to the subject's anatomical surface, with the acoustically transmissive material providing a high fidelity acoustic path between the subject's surface and the emissive face of the ultrasound probe.

An ultrasound protocol may be initiated following positioning, orientation and adjustment of the framework structure, probe mount and ultrasound probe. In one embodiment, an associated ultrasound monitoring system having a display is operated to identify and locate a probe illumination area, an operator manipulates the ultrasound probe and/or probe mount to match the probe illumination area with a target marked on the display, and the operator then locks the probe and/or probe mount into place. The ultrasound monitoring system may be programmed to alert the subject, or an operator, if the probe illumination area strays from the target, or if or when the probe needs to be repositioned and the target re-acquired. Various types of protocols for automated target location and station-keeping may be implemented.

Many of the ultrasound monitoring systems and components described in detail below are intended for use in cranial ultrasound monitoring applications. It will be appreciated, however, that similar systems and components may be designed, and used, for monitoring other physiological sites. Framework components or other types of mounting systems may, for example, be designed for mounting around a subject's neck for monitoring carotid artery blood flow, for example, or for mounting around a subject's torso or limbs for other ultrasound monitoring applications. Similar types of adjustable probe housings, probe mounts and interface components may likewise be used with other types of framework components and mounting systems.

### BRIEF DESCRIPTION OF THE FIGURES

FIG. 1 illustrates components of one embodiment of a framework structure for mounting on a subject's cranium, an ultrasound transducer framework and mounting structure, a transducer interface component and an ultrasound transducer for mounting on the framework structure.

FIG. 2A illustrates an exemplary headband and ultrasound transducer framework structure ready to be mounted on a subject's skull, and FIG. 2B illustrates the ultrasound transducer framework and mounting structure mounted on a subject's skull.

FIG. 3 shows a schematic side view of one embodiment of an ultrasound transducer framework and mounting structure for adjusting the position of the transducer housing and the transducer.

FIG. 4 shows a schematic side view of another embodiment of an ultrasound transducer framework and mounting structure for adjusting the position of the transducer housing and the transducer.

FIG. 5 illustrates a kit containing single use and/or single patient components of an ultrasound monitoring system of the present invention.

FIGS. 6A-6D illustrate one embodiment of a probe interface component suitable for use as a single use interface component, the mounting of an ultrasound transducer probe into the interface component, and the mounting of the interface component into an adjustable transducer mount installed on a framework structure. FIGS. 6A and 6B show an ultrasound transducer probe being mounted in a transducer interface component; FIG. 6C schematically illustrates the probe and interface component assembly being mounted in a probe mount provided on a framework structure, and FIG. 6D illustrates the probe and interface component assembly mounted in the probe mount and ready for positioning on an anatomical surface of a subject.

FIGS. 7A-7B illustrate a removal sequence of an ultrasound probe from the probe interface component illustrated in FIGS. 6A-6D. FIG. 7A illustrates removal of a retention band from the probe interface component to release the probe, and FIG. 7B shows removal of the probe from the interface component following removal of the retention band. The probe interface component illustrated here is designed as a single use component.

FIG. 8A schematically illustrates a perspective view of another embodiment of a framework structure for mounting an ultrasound transducer mounting mechanism and ultrasound transducer.

FIG. 8B schematically illustrates a perspective view of a framework structure similar to that shown in FIG. 8A and an underlying headband component having mating element(s) for mounting the framework structure.

FIG. 9A is a schematic illustration of a framework structure of FIG. 8 (in part) with a probe mounting structure and ultrasound probe adjustably mounted on the framework structure; and FIG. 9B is an exploded diagram illustrating the components of the framework and probe mounting structure illustrated in FIG. 9A.

FIG. 10A is a schematic illustration of an ultrasound probe housing and interface component in an assembled condition; FIG. 10B is a schematic illustration of the ultrasound probe housing and interface component in an unassembled condition.

FIGS. 11A-11E illustrate an exemplary sequence for placing and positioning a cranial framework structure and ultrasound probe(s) on a subject's skull and targeting of the ultrasound probe(s) to a desired cranial target location. Specifically, FIG. 11A illustrates the framework structure positioned for mounting on a subject's cranium; FIG. 11B illustrates the framework structure positioned on a subject's cranium; FIG. 11C illustrates initial operation of an associated ultrasound monitoring system to locate the ultrasound probe illumination area; FIG. 11D illustrates manipulation of the ultrasound probe to match the probe illumination area with a target; and FIG. 11E illustrates matching of the probe illumination area with the desired target.

### DETAILED DESCRIPTION

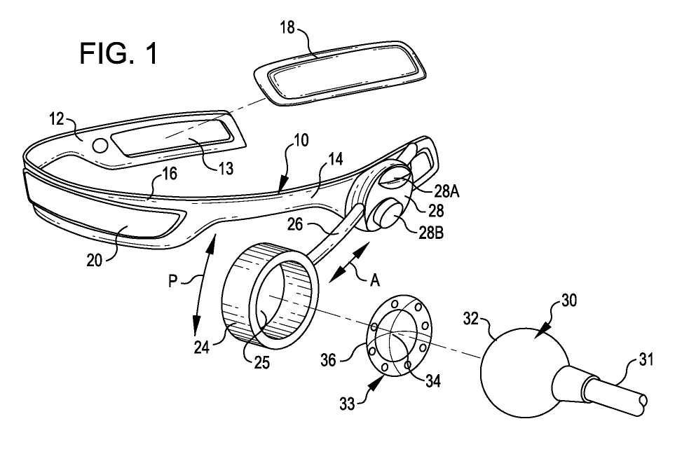

In one embodiment, illustrated schematically in FIG. 1, a framework structure for use with ultrasound monitoring systems requiring interface of an ultrasound probe with a subject's anatomy, such as an anatomical surface at a cranial window, (e.g., at a temporal window), comprises a generally U-shaped frame member 10 sized and configured for placement on a subject's skull. Frame member 10 comprises two framework legs 12, 14 positioned opposite one another for placement on opposite sides of a patient's skull and a connecting member 16 positioned to provide a bridge between the framework legs. In some embodiments, connecting member 16 may be configured to contact and generally conform to the shape of a subject's forehead. In some embodiments, the frame member 10 may be configured for positioning connecting member 16 adjacent to or contacting a subject's forehead; in alternative embodiments, frame member 10 may be configured for positioning connecting member 16 adjacent to or contacting the top of a patient's skull. Frame member 10 is preferably constructed from a substantially rigid or semi-rigid material that is lightweight, resilient and flexible, permitting movement (opening) of the legs of the U-shaped framework for placement on a subject's skull, and firm retention of the frame member on the skull once positioned. The frame member may be constructed from a variety of resilient materials; suitable metallic, thermoplastic and polymeric materials are well known in the art. Various contoured features, apertures, decorative and/or identification features, and the like, may be provided in association with or incorporated in the frame member. It will be appreciated that frame members may be configured as generally U-shaped structures, as described and illustrated. Alternatively, frame members may be provided as generally round or oval structures, or in other configurations for mounting on various anatomical locations, such as the neck, limbs, the torso, and the like.

The frame member may be constructed having solid surfaces, or grooved, perforated or ridged surfaces may be provided. In one embodiment, frame member 10 may comprise one or more cut-outs 13 for receiving insertable and/or detachable mounting elements. In the embodiment illustrated in FIG. 1, mounting element(s) 18 (shown before mounting in a framework leg cut-out) and 20 (shown mounted in a framework connecting member cut-out) fit snugly in mating cut-outs in frame member 10. The mounting elements may be provided as single use and/or patient specific elements, and they may project from the surface of the frame member on an inside and/or outside surface. They may be constructed from a material that is adherent or semi-adherent to a patient's skin surface, such as a rubbery or pliable material that is comfortable against the skin of the subject. They may also be constructed from a material that is adherent or semi-adherent to the surface of an (optional) underlying band that contacts the subject's skin directly. Mounting element(s) 18, 20 may be customized, or customizable, for specific subjects so that, when mounted in a framework structure, the assembly is easily identified with a particular subject. Framework 20 and/or mounting element(s) 18, 20 may also be customized, or customizable, for specific ultrasound operating systems, transducers, probes, protocols, or the like.

The system of FIG. 1 also illustrates a probe mount 24 mounted on a mounting structure such as arm 26 that interfaces with and may be controlled by adjustment mechanism 28 mounted on frame member 10. In this embodiment, probe mount 24 may be fixed with respect to arm 26, and arm 26 may be adjustable both pivotably (along a path P) and axially (along a path A) with respect to adjustment mechanism 28 and frame member 10. Multiple actuating controllers may be provided. Actuator 28A may, for example, when actuated, allow sliding of arm 26 (and probe mount 24 and the ultrasound probe mounted therein) along axis A, while actuator 28B may, for example, when pushed or actuated, provide pivoting of arm 26 (and probe mount 24 and the ultrasound probe mounted therein) along path P. In some embodiments, arm 26 may be moved axially and pivoted simultaneously by activation of both actuators simultaneously, or by activation of a third actuator. In some embodiments, arm 26 and/or adjustment mechanism 28 may be detachable from frame member 10.

In some embodiments, another actuating controller may be provided that allows movement of arm 26 (and probe mount 24 and the ultrasound probe mounted therein) along a path toward and away from a subject's skull surface, e.g. along an axis substantial orthogonal to both path A and path P. In some embodiments, arm 26 may be biased or biasable generally toward the opposite framework leg to promote contact of the probe and/or probe mount and/or probe interface component with the subject's anatomy. In some embodiments, adjustment mechanism 28 may be slidable on framework member 10, or removable from and positionable at different locations on framework member 10 to provide additional adjustment flexibility. These adjustment mechanisms allow an operator, or a subject, to position the probe housing (and the ultrasound probe and transducer(s) mounted therein) in a variety of positions on a patient's anatomical surface(s), e.g., skull. These adjustment features, or additional features, may also allow an operator, or a subject, to adjust the contact pressure of the probe mount, or the probe, or an interface component, against the patient's anatomical surface(s).

A locking device is preferably provided for locking and securing the position of the ultrasound probe mount (and the ultrasound probe and transducer(s) mounted therein) securely in a selected position. Many different types of locking mechanisms may be used. In one embodiment, a locking device may comprise an actuator that locks the axial and/or pivotal position of the probe mount separately or in a unified fashion following positioning. In another embodiment, a locking mechanism may comprise a squeeze clamp that releases by mechanically squeezing the clamp to allow positioning of an arm and/or probe mount and, when released, locks the position of the arm and/or probe mount.

Ultrasound probe 30 is preferably removably mountable in probe mount 24. Probe 30 may comprise a single element ultrasound transducer; it may comprise a standard TCD probe; it may comprise a one or two dimensional ultrasound transducer array; it may comprise a diagnostic and/or scanning and/or therapeutic transducer; and it may incorporate other types of ultrasound transducer or probe assemblies that are known in the art. Several types of ultrasound transducers, transducer combinations and arrays are described in U.S. Pat. No. 7,547,283, the disclosure of which is incorporated herein by reference in its entirety. It will be appreciated that acoustic transducer arrays having various configurations and structures are known in the art and may be useful for various applications. Acoustic transducer arrays suitable for use in the present invention are generally thin and may comprise a single layer or thickness of transducer elements. Stacked, multiple layer transducer cells, or elements, may be used for some applications. Transducer elements or cells may be arranged on a single plane to form a generally flat, planar array, or they may be arranged to form a curved or a geometrically stepped array.

Ultrasound probe 30 illustrated in FIG. 1 has a housing with a generally curved, spherical outer configuration and a curved acoustic emission surface 32. Ultrasound probes having other surface configurations may be used as well; it will be appreciated that ultrasound probes and housings having various surface configurations and structures are known in the art and may be useful for various applications. Ultrasound probe 30 may be in operable communication with a power source and/or controller (not shown in FIG. 1) via, e.g., cable 31 or using various types of wireless protocols.

Probe interface member 33 may be provided as an interface between an acoustic emission surface 32 of ultrasound probe 30 and a subject's anatomical surface (e.g. skin, skull). In the embodiment illustrated in FIG. 1, probe interface member 33 has a flexible, pliable, acoustically transmissive interface portion 34 provided centrally and a mounting portion 36 located generally at the periphery of interface portion 34. In operation, probe interface member 33 may be inserted in probe mount 24 and positioned within the probe mount 24 with acoustically transmissive interface portion 34 exposed through a window or port 25 in probe mount 24. Ultrasound probe 30 may then be positioned in probe mount 24 with its acoustic emission surface 32 positioned in contact with one surface of acoustically transmissive interface portion 34. Alternatively, probe interface member 33 may be mounted on the acoustic emission surface 32 of the ultrasound probe 30, and the combination may be inserted and positioned within the probe mount 24. In either case, a surface of acoustically transmissive interface portion 34 is exposed through window 25 in probe mount 24 and, when the probe mount and the installed ultrasound probe are positioned in preparation for conducting an ultrasound protocol, a high fidelity acoustic path is provided, through interface portion 34, between the emissive probe face 32 and the subject's anatomical surface at the desired location.

Transmissive interface portions having different sizes, configurations, thicknesses, stand-off dimensions, transmissive properties, and the like, may be provided for various diagnostic and monitoring purposes and for use with different types and configurations of ultrasound probes and transducer emission surfaces. Probe interface member 33 is generally provided as a single use component to ensure high fidelity acoustic transmission between the probe emission surface 32 and the subject's anatomical surface and may be packaged as a clean or sterile component.

Probe mount 24, transducer interface member 33 and ultrasound probe 30 are sized and configured such the components may be assembled and disassembled easily and conveniently and, when the components are assembled, they have a snug fit and are stably positioned relative to one another. Interface member 33 may have a mating configuration with complementary surfaces of probe mount 24 or may be mountable in probe mount 24, and/or on the acoustic emission surface 32 of ultrasound probe 30, to provide stable positioning of the interface member and transducer, and to provide reliable and consistent contact between a subject's anatomical surface (e.g., skin, skull), interface member 33, and acoustic emission surface 32 of ultrasound probe 30. This stable positioning may be provided, for example, using a press-fit or another secure and stable system for mounting interface member 33 to the probe mount and/or probe, and for mounting the probe to the probe mount.

In another embodiment, acoustically transmissive gels and other substances may also be used to provide or enhance the acoustic path between an emissive surface of an ultrasound probe and a subject's anatomical surface, whether or not a transducer interface member is used. In one embodiment, an ultrasound probe may be mounted directly in a probe mount, for example, with the acoustically emissive face of the probe exposed through a window or port in the probe mount. An acoustic path between the probe face and the subject's anatomical surface may be established using acoustically transmissive gel. In yet another embodiment, an acoustic path may be provided between a probe face and the subject's anatomical surface using another acoustically transmissive element, such as a “pad” or volume of acoustically transmissive material provided having a size and configuration suitable for establishing, and maintaining, an acoustic path between the emissive probe surface and a subject's anatomical surface. One or both contact surfaces of an acoustically transmissive “pad” component may have an adhesive or bonding layer providing securely detachable positioning of the pad component on the emissive face of the probe and/or the subject's surface. Suitable acoustically transmissive pad components may be provided in a variety of configurations, geometrical shapes, thicknesses, and the like, and may provide a variety of acoustic transmission properties.

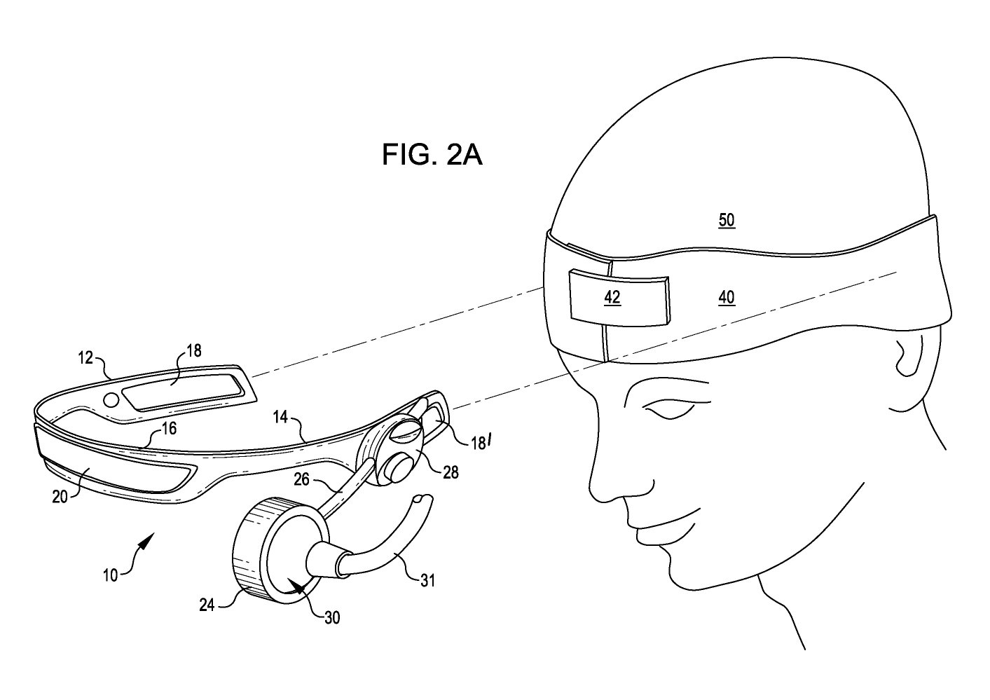

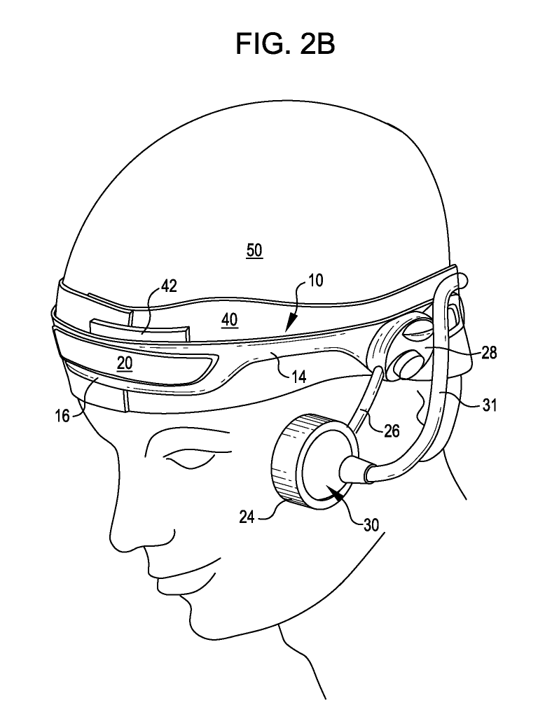

An underlying comfort band that fits securely and comfortably around a subject's anatomical surface, such as the skull, may be provided for patient comfort and to positively position and retain the frame member in a stable position on the subject. FIG. 2 shows a subject having a band 40 mounted on his skull 50. The band is generally adjustable or may be modified to fit comfortably and securely around a subject's anatomical surface, e.g., skull. Band 40 is flexible and may be elastic, and it may be provided with one or more straps, fasteners, or the like to securely fasten the band, in a comfortable position, on a subject's anatomical surface. Suitable bands may be constructed from generally soft and pliable materials such as natural and synthetic fabrics, rubbery materials, and the like, and may be constructed as a single piece or in multiple pieces. In one embodiment, the band component comprises an elastic fabric component having a fastening mechanism 42 in proximity to each terminal end, such as a hook and loop fastener (e.g., Velcro®), to fasten terminal ends of the band to one another. Band components are generally provided for use by individual patients and may be used for a single or multiple ultrasound operation(s). They may be customized to individual users, or individual categories of users, or for use in connection with various ultrasound protocols and at various anatomical sites by providing customized sizes, configurations, colors, decorations, fasteners, fastener locations, and the like. It will be appreciated that similar types of bands having different configurations and dimensions may be provided for mounting on anatomical sites other than a subject's skull.

FIGS. 2A and 2B show a schematic diagram of a subject wearing a band 40 with a framework member 10 and a single ultrasound probe 30 and probe mount 24 in position both prior to being mounted on the subject's skull (FIG. 2A) and following mounting over the band on the subject's skull (FIG. 2B). Framework member interfaces 10 with the underlying band 40 to comfortably and securely mount the framework 10 in position for acquiring ultrasound data. Ultrasound probes are generally in communication with power source(s) and control system(s) for administering ultrasound interrogation and/or detection protocols, collecting ultrasound data or monitoring a desired target site by administering ultrasound protocols over a period of from several minutes to several hours to several days or more. The ultrasound components described herein may be used with many different types of ultrasound control systems, probes, protocols, and the like, including diagnostic, imaging and therapeutic ultrasound systems, probes and protocols.

Framework member 10 is mounted over the band 40 and incorporates mounting interfaces 18, 18′ and 20. Legs 12, 14 of the framework member are positioned on generally opposite sides of the subject's skull, while cross member 16 is positioned generally across the subject's forehead. Probe mount 24 and ultrasound probe 30 are adjustably positioned so that a probe interface member is positioned in proximity to and generally contacts, directly or indirectly (e.g., through an acoustically transmissive gel or pad), acoustic emission surface of the ultrasound probe and the subject's surface to provide an acoustic transmission path between the ultrasound transducer and the anatomical surface. Adjustment of the probe mount 24 and ultrasound probe 30 in two- and/or three-dimensional space is provided as described above, allowing positioning of the ultrasound probe with respect to a desired anatomical surface in accordance with each subject's individual anatomy and the requirements of various ultrasound systems and protocols. Once the ultrasound probe and probe mount are positioned appropriately for an ultrasound protocol, they may be locked in place to maintain proper positioning. The probe cable(s) may be led away from the transducer and housing, as shown in FIG. 2B, and may interface or interlock with cable retention or positioning systems provided in connection with the framework member, the band, or both.

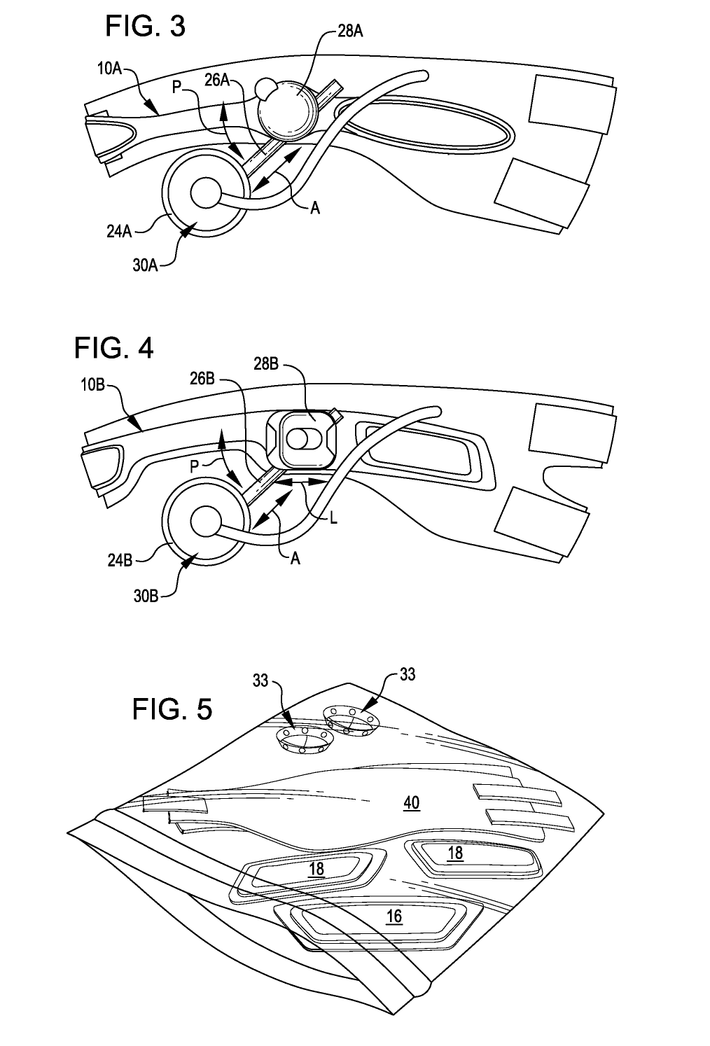

FIGS. 3 and 4 schematically show alternative embodiments for adjusting the probe mount and the probe with respect to the framework component. In the embodiment illustrated in FIG. 3, ultrasound probe 30A is mounted in probe mount 24A. Arm 26A is mounted on probe mount 24A and received through adjustment mechanism 28A mounted on framework member 10A. In this embodiment, adjustment mechanism 28A provides adjustment of arm 26A, probe mount 24A and ultrasound probe 30A along linear path A. Adjustment mechanism 28A may additionally provide adjustment of arm 26A, probe mount 24A and ultrasound probe 30A along a pivoting path P. In addition, probe mount 24A may be gimbaled with respect to arm 26A to provide tilting and angular adjustment of the housing and/or an installed ultrasound probe 30A along a variety of rotational and/or spherical paths with multiple degrees of freedom. A locking device may be actuated to lock axial movement, pivoting and/or rotation of the transducer housing following positioning, and to securely maintain the ultrasound probe mount and ultrasound probe in a selected position.

FIG. 4 shows a similar arrangement in which ultrasound probe 30B is mounted in probe mount 24B. Arm 26B is mounted on probe mount 24B and received through adjustment mechanism 28B mounted on framework member 10B. In this embodiment, adjustment mechanism 28B, in addition to providing axial and pivoting adjustment of arm 26B along axial path A and pivoting path P, provides adjustment of arm 26B, probe mount 24B and ultrasound probe 30B along a lateral path L. In addition, probe mount 24B may be gimbaled with respect to arm 26B to provide tilting and angular adjustment of the housing and probe with multiple degrees of freedom about an axis generally orthogonal to the contact surface of the probe face, or the interface component, with the subject's surface. One or more locking device(s) may be provided to lock axial, lateral, pivoting and angular adjustment of the probe mount following positioning, and to securely maintain the probe mount and ultrasound probe in a selected position.

The framework embodiments illustrated in FIGS. 1, 2A, 2B, 3 and 4 are illustrated providing a single adjustable arm and probe mount, and are suitable for use with ultrasound systems and protocols utilizing a single ultrasound probe. It will be appreciated that multiple ultrasound probe mounts, positioning arms and adjustment features may be provided for positioning multiple probes on one or more patient anatomical surfaces simultaneously or at different times. In some embodiments, bilateral probe mounts may be provided on bilateral positioning arms mounted to adjustment mechanisms provided on each of multiple framework legs. This allows, for example, ultrasound protocols and monitoring of separate and distinct target sites within a target inspection area, such as the CNS, simultaneously. The operation of multiple probes may be coordinated by a common controller that communicates with and collects data from each of multiple probes.

Framework components may be provided, and used, as reusable or single use components, or they may be provided or customized for individual subjects, or for various specific types of ultrasound transducer probes and protocols. The framework components may be configured to conform to individual subject's anatomical surface (e.g., skull) and provided as a custom-fitted component, or framework components may be designed to fit multiple skull sizes and configurations. For some applications, a framework component with one or more probe mount(s), arm(s) and adjustment mechanism(s) are assembled as a kit and provided as reusable components. Probe interface components providing a high fidelity acoustically transmissive path between an acoustic emission surface of a transducer and the subject's anatomical surface are generally provided as single use, single monitoring period components. Probe(s) having different ultrasound interrogation and/or detection capabilities and functionalities that mate with the probe mount(s) may be provided separately and interface with appropriate power source(s), controller(s), ultrasound data acquisition system(s), monitoring system(s), display(s), data storage device(s), and the like.

Components such as a comfort band and/or transducer interface components and/or framework mounting elements may be provided as single use components and may be packaged as a kit, as illustrated in FIG. 5. In one embodiment, kits of the present invention may comprise one or more components selected from the group consisting of band component(s) 40, mounting elements 16, 18, and transducer interface member(s) 33. Any of these components may also be packaged, and distributed, singly or in multiple component kits. Any or all of these components may be packaged as clean or sterile components.

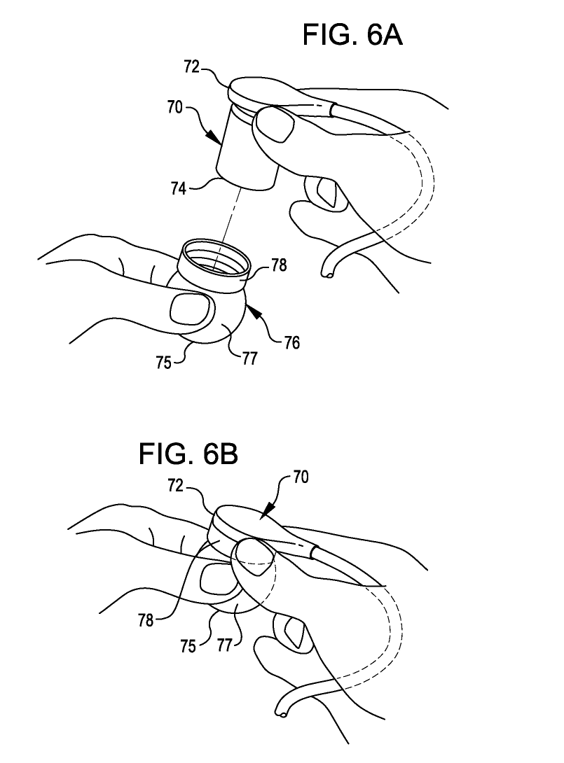

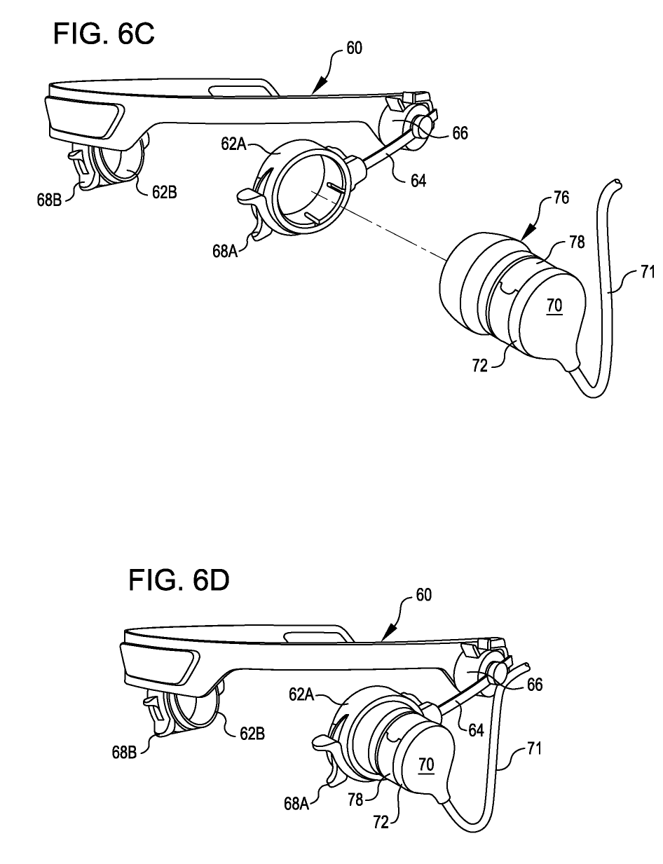

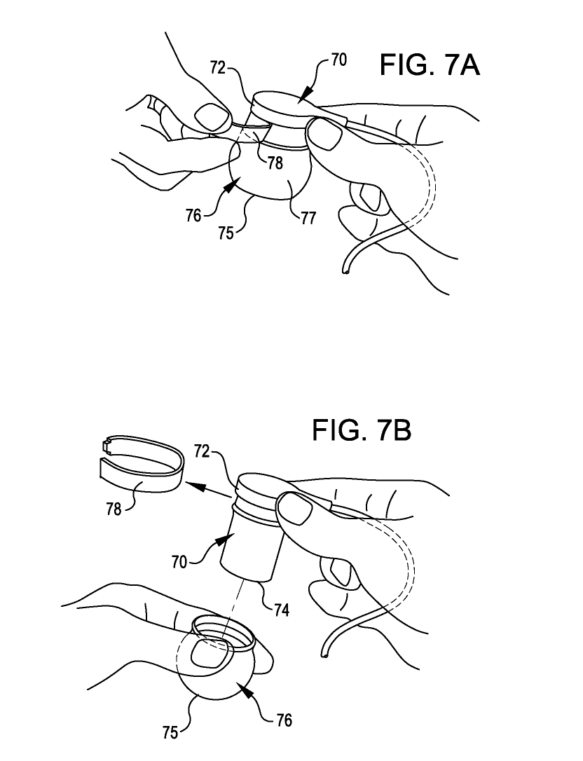

FIGS. 6A-6D and 7A-7C illustrate alternative embodiments of a framework member, probe mount, ultrasound probe and probe interface components of the present invention. In this embodiment, framework member 60 (FIGS. 6C, 6D) is generally U-shaped and has probe mounts 62A, 62B mounted on arms 64 extending from adjustment mechanisms 66 mounted on each of the two framework legs. The interior dimension(s) of probe mounts 62A, 62B are preferably adjustable using clamping mechanisms 68A, 68B. In one embodiment, squeezing the projections of clamping mechanisms 68A, 68B toward one another enlarges the interior dimension(s) of the probe mount(s), permitting insertion and mounting of an ultrasound probe and probe interface component within the interior of the probe mount. Releasing the adjustment mechanisms clamps and stably holds the probe interface component and probe housing within housing 62A, 62B, with the acoustically emissive probe face positioned for carrying out an ultrasound protocol.

The ultrasound probe housing may have a variety of external configurations. A generally spherical probe housing 30 is shown in FIG. 1. FIG. 6A shows a generally cylindrical ultrasound probe housing 70 having an enlarged shoulder or rim 72 that may function as a mechanical stop during mounting of the probe housing in an interface member. Probe interface member 76, in this embodiment, comprises a probe housing 77 providing an interior cavity sized to receive at least a portion of probe 70, and a releasable retention member 78. The interior space formed by the probe housing 77 may correspond generally to the exterior configuration of an ultrasound probe for use with the interface member 76. As shown in FIG. 6B, at least a portion of ultrasound probe 70 is inserted into and fits snugly into the interior space of interface member 76, with an acoustically emissive portion 74 of probe 70 in proximity to and/or contacting acoustically transmissive elements or materials associated with interface member 76 to provide an acoustically emissive end face 75. The outer surface configuration of the interface member 76 may have a generally curved, rounded or spherical configuration, as shown in FIGS. 6A and 6B. Retention member 78 of interface member 76 may interact with enlarged shoulder 72 or another portion of the probe housing to mechanically couple the interface member to the probe housing during use. In one embodiment, interface member 76 is provided as a single use component that is stably mountable on an ultrasound probe.

In the embodiment illustrated in FIGS. 6A-6D, the end face 74 of probe housing 70 incorporates the acoustically emissive face 74 of the probe and the corresponding end face, or end region 75 of interface member 76 comprises an acoustically transmissive material that provides an acoustically transmissive path between the acoustically emissive face 74 of the probe and an anatomical surface of the subject and functions as an acoustic coupler. Suitable acoustically transmissive materials are well known in the art. In one embodiment, the acoustic coupler comprises a thermoplastic elastomer, such as an oil-enhanced or gelatinous thermoplastic elastomer. Suitable materials are described, for example, in U.S. Patent Publication 2005/0215901 A1. In one embodiment, such a material forms the end face 75 of interface member 76 and, when probe housing 70 is mounted in interface member 76, the acoustically emissive surface 74 of the inserted probe intimately contacts the acoustic coupler forming end face 75 of the interface member.

FIGS. 6A and 6B illustrate mounting of probe housing 70 having an acoustically emissive face 74 in interface member 76. When mounted, probe housing 70 is stably and securely held in interface member 76 using, for example, a mechanical securing arrangement, such as interacting rims, grooves, and the like that may provide a press-fit. The probe/interface assembly is then stably mounted in probe mount 62A, 62B of framework member 60 using adjustment mechanisms 68A, 68B, as illustrated in FIGS. 6C and 6D. Probe mounts 62A and 62B are adjustable along multiple paths and in multiple dimensions, as described above, to position the probe housing and probe on a subject's anatomical surface, as desired. In the configuration illustrated in FIGS. 6A-6D, interface member 76 has a curved and partially spherical surface that contacts a mating curved and partially spherical surface on an interior surface of probe housing 62A when interface member 76 is mounted in the probe housing 62A. Movement and adjustment of these mating, curved, partially spherical surfaces relative to one another provides additional tilting and angular adjustability of the probe face along rotational and/or spherical paths with multiple degrees of freedom. A locking device may be actuated to lock axial movement, pivoting and/or tilting and angular adjustment of the transducer housing following positioning, and to securely maintain the ultrasound housing and ultrasound probe in a selected position.

FIGS. 7A and 7B illustrate one exemplary mechanism for conveniently removing the probe housing 70 from interface member 76, allowing re-use of the probe and disabling the interface member to prevent re-use of the interface member. Upon completion of a desired ultrasound protocol, the assembly comprising probe housing 70 and interface member 76 is removed from the transducer mount 62A (by adjustment, for example, of clamping mechanism 68A). FIG. 7A shows the release of releasable retention member 78, provided as a peelable retention strip, from interface member 76, releasing probe housing 70 from the interface member and allowing it to be removed from interface member 76, as shown in FIG. 7B. In the embodiment illustrated in FIG. 7A, releasable retention member 78 is formed as a peel-away structure with a grasping tab to facilitate removal of the retention member to release the transducer from the interface member. Other types of releasable retention systems may be used alternatively or additionally. Once the releasable retention member has been released, or removed, the transducer is easily and conveniently removable from interface member 76 and, in many embodiments wherein the interface member is provided as a single use, disposable component, the used retention member 78 and interface member 76 may be discarded. In the interface member embodiment illustrated in FIGS. 7A and 7B, the interface member is desirably rendered unusable following use and removal from the ultrasound probe.

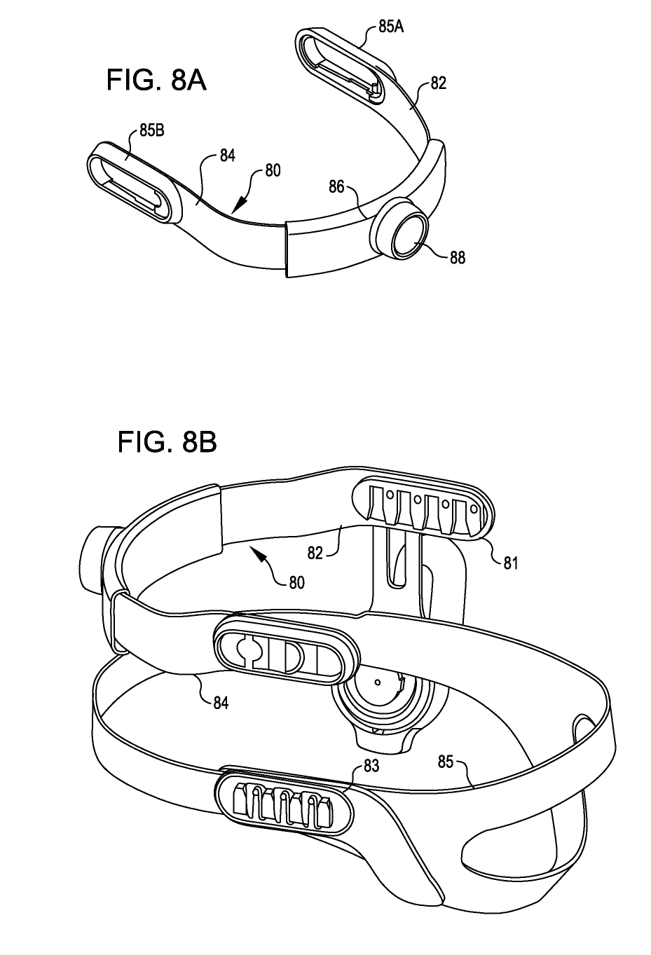

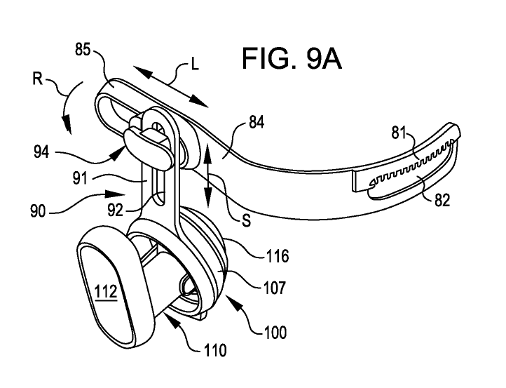

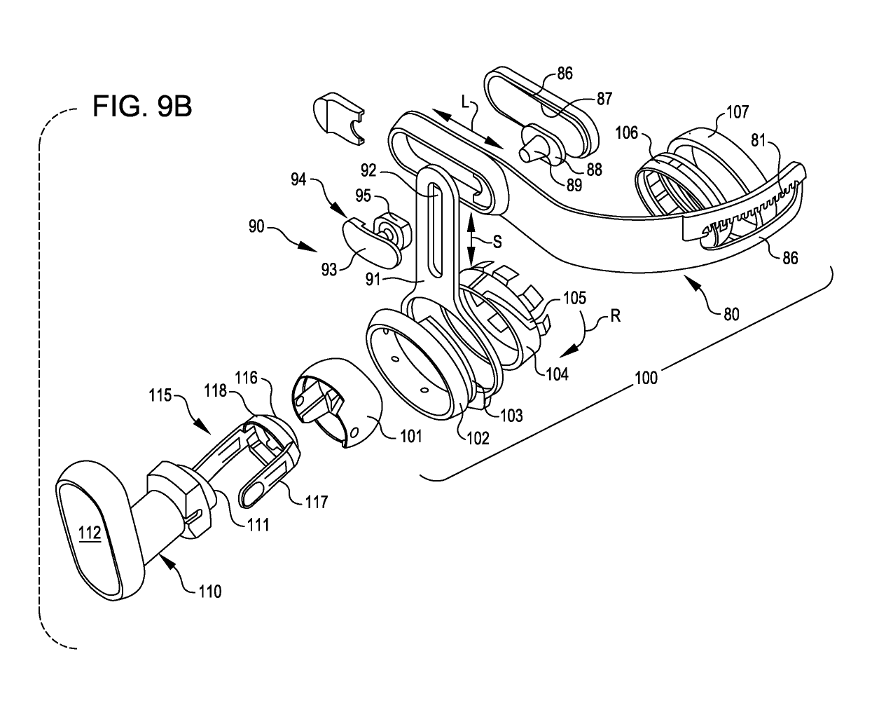

FIG. 8A schematically illustrates another embodiment of a framework component of the present invention and FIG. 8B schematically illustrates a band and framework component, and the mating interaction of the band and framework to provide stable mounting of the framework component to the band. Framework component 80 is generally U-shaped frame member sized and configured for placement on a subject's anatomical structure, such as a skull. Frame member 80 comprises two framework legs 82, 84 positioned opposite one another for placement on opposite sides of a patient's skull and a connecting member 86 positioned to provide a bridge between the framework legs. In the embodiment illustrated, connecting member 86 incorporates an adjustment mechanism 88 for adjusting the size and/or configuration of the connecting member and to assist in customizing the fit of the framework member to a variety of subjects and/or anatomical surfaces. In the embodiment shown, adjustment mechanism 88 may be provided as a rotatable adjustment knob that interacts with a toothed structure 81 (shown in FIGS. 9A and 9B), or another adjustment structure on the underlying framework structure, to expand or contract the dimensions of the connecting member, thereby positioning the framework legs closer together or further apart. The frame member may be constructed from a variety of resilient, elastic materials; suitable metallic, thermoplastic and polymeric materials are well known in the art. Various contoured features may be provided. It will be appreciated that similar types of frame members may be configured as generally U-shaped structures, or as generally round or oval structures, or in other configurations, for mounting on other anatomical locations, such as the neck, limbs, the torso, and the like.

Framework component 80 may have associated mounting structures 85A, 85B for receiving a probe mount and adjustment mechanism 90. Mounting structures 85A, 85B may be formed integrally with the framework component or may be provided as separate components mountable on and, optionally, adjustable with respect to framework component 80. In one embodiment, mounting structures 85A, 85B may be laterally and/or axially adjustable on framework legs; in another embodiment, mounting structures 85A, 85B may alternatively or additionally be rotatable with respect to the framework legs.

FIG. 8B illustrates an embodiment in which framework component 80 additionally has mounting structures 81 provided on an interiorly facing surface of framework legs 82, 84. In the embodiment illustrated in FIG. 8B, mounting structures 81 interact and mate with complementary mounting structures 83 provided on an underlying band 85. Band 85 has substantially flexible portions to provide a secure and comfortable fit on a patient's anatomical surface, such as a skull. Band 85 may be elastic and may be adjustable by means of one or more straps, fasteners or the like to provide different size and configuration options. Band 85 additionally comprises at least one semi-rigid element or stiffener, which may be provided as mounting structure(s) 83. The incorporation of one or more semi-rigid element(s) or stiffener(s) on band 85 desirably enhances the structural integrity of the band and, when the stiffener element(s) additionally incorporate mounting structure(s), these features facilitate mounting and installation of the frame member over the band. In the embodiment illustrated in FIG. 8B, framework mounting structures 81 are slotted, forming a plurality of grooves and tabs arranged in a side-by-side relationship. Band mounting structures 83 have a complementary arrangement of grooves and tabs for slidably engaging the grooves and tabs provided on framework mounting structure 81.

Upon engagement, the complementary framework mounting structures 81 and band mounting structures 83 provide stable mounting of the framework structure to the band. The complementary mounting structures also provide adjustable positioning of the framework structure relative to the band by alignment of the complementary grooves and tabs in more forward or rearward positions to accommodate close fitting to anatomical structures having different sizes and shapes. In one scenario, a band may be positioned on a subject's anatomical surface (e.g., skull) and the grooves and tabs of the mounting structure of the framework may be aligned with and mounted on the complementary grooves and tabs of the mounting structure provided on the band, as appropriate, to provide a generally loose fit of the framework structure over the underlying band and subject's anatomical structure. Adjustment knob 80 may then be manipulated to further adjust (e.g., tighten) the framework structure over the band to provide a comfortable, yet close fit of the framework structure over the band and on the underlying anatomical structure.

FIG. 9A illustrates a probe mounting and adjustment mechanism 90 mounted on mounting structure 85 on framework component 80; FIG. 9B illustrates an exploded view showing individual components of the probe mount and adjustment mechanism 90. In the embodiment illustrated in FIGS. 9A and 9B, probe mount and adjustment mechanism 90 is mountable on mounting structure 85 and is slidable along two different linear paths. Probe mount 90, when installed on mounting structure 85, is slidable along a longitudinal axis of mounting structure 85, along a linear adjustment path parallel to arrow L. Probe mount 90 is also slidable along a longitudinal axis of slot 92 in arm 91, along a linear adjustment path parallel to arrow S. Slot 92 is illustrated positioned and mounted generally orthogonal to the longitudinal axis of mounting structure 85, and probe mount 90 is thus adjustable along generally orthogonal linear paths parallel to arrows L and S to position the probe at desired locations on a subject's anatomical surface(s).

In alternative embodiments, the configuration of the mounting structure 85 and slot 92, and thus the movement of the probe mount along paths corresponding to L and S may be oriented in a non-orthogonal relationship. In addition, while paths L and S are illustrated as straight line linear paths, it will be appreciated that linear adjustment paths, in certain embodiments, may have a curved profile or a may incorporate multiple axial and/or curved paths. Adjustment of probe mount 90 along these adjustment paths may be in a single or two dimensional linear (e.g., straight line or curved) path, or may additionally incorporate an additional i e.g. toward and away from the framework structure 80. Thus, adjustment of probe mount 90 along a linear (e.g., straight line or curved) path may additionally involve adjustment of the probe mount in another dimension toward and/or away from the framework.

In some embodiments, mounting structure 85 may be rotatable or pivotable and lockable in multiple orientations on framework structure 80 to change the orientation of linear path L, providing additional and alternative adjustment configurations. In some embodiments, slot 92 provided in arm 91 may have different orientations, changing the direction of linear path S and providing additional and alternative adjustment configurations. In yet additional embodiments, arm 91 may comprise multiple slots oriented at different angles to provide multiple axial adjustment options and paths of travel for transducer housing and adjustment mechanism 90.

In the embodiments illustrated in FIGS. 9A and 9B, transducer mount 90 is mountable on mounting structure 85 by means of a lockable fastener 94. In this embodiment, cap 86 having a groove 87 retaining sliding member 88 is mounted to the framework structure 80 and/or to mounting structure 85. A projection 89 of sliding member 88 extends through a slot in framework structure 80 and mounts to arm 91 using, for example, locking fastener 94. In the illustrated embodiment, locking fastener 94 comprises an exterior tab 93 sized and configured for positioning on an external side of arm 91 for manipulation by an operator, and a spaced apart insertion member 95 insertable (in at least one orientation) through slot 92 and fastenable on projection 89 of sliding member 88. Locking fastener 94, as shown, may be positioned with fastening member 95 aligned with slot 92 to insert insertion member through slot 92 for fastening on projection 89 and then rotated to lock the transducer mount 90 in place on mounting structure 85 and/or framework structure 80.

In some embodiments, probe mount 90 may be adjustable along at least two linear paths and also along a rotational path R, with the central axis of locking fastener 94 forming the axis of rotation. When locking fastener 94 is in an unlocked condition, transducer mount 90 may be adjustable along at least two linear paths and additionally along a rotational path R with respect to the framework structure (and a subject's anatomical surface(s)). Adjustment of locking fastener 94 to a locked condition may effectively and simultaneously stabilize, and/or lock, probe mount 90 in a desired position along at least two different linear paths and at least one rotational path. This embodiment thus provides adjustment of a transducer mount along at least two linear paths and at least one rotational path and provides a fastening mechanism that serves as a common locking mechanism for each of the adjustment paths. In another embodiment, mounting structure 85 may be rotatable, and lockable in a variety of orientations to provide rotational adjustment of an associated probe mount 90.

Probe mount 90 may additionally comprise, or receive, components for interfacing with, securing and orienting an ultrasound probe within the probe mount and, optionally, provide additional adjustment of an ultrasound probe with respect to the framework structure and a subject's anatomical surface(s). In the embodiments illustrated in FIGS. 9A 9B, 10A and 10B, an ultrasound probe housing 110 incorporating an ultrasound transducer operated and controlled, at least in part, by an external ultrasound controller (not shown) has an acoustically emissive face 111 and an enlarged handle 112. Probe housing 110 may be connected or connectable to an external system via cables, wireless protocols, and the like, as is well known in the art.

In the embodiments illustrated in FIGS. 9A and 9B, ultrasound probe housing 110 is mountable in a complementary receiving portion 100 of probe mount 90. Receiving portion 100 may be configured and designed to securely retain probe housing 110 (or a portion of probe housing 110), and receiving portion 100 may be adjustable or non-adjustable with respect to other elements of probe mount 90, framework structure 80, and/or anatomical surfaces of a subject, as described in greater detail below.

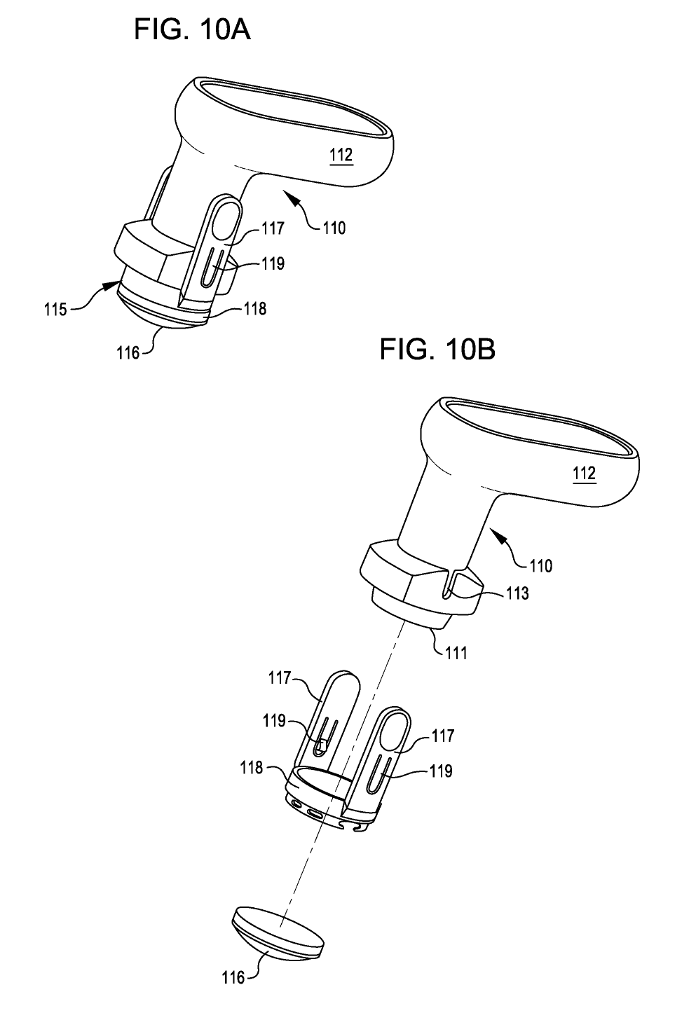

In embodiments that are preferred for certain applications, probe housing 110 interfaces with a probe interface component 115 shown in FIGS. 10A and 10B that mounts to (e.g., over) probe housing 110 and provides an acoustically transmissive interface 116 comprising an acoustically transmissive material that provides a transmissive path between the acoustically emissive face 111 of an ultrasound probe mounted in housing 110 and an anatomical surface of the subject and functions as an acoustic coupler. Suitable acoustically transmissive materials are well known in the art. Acoustic couplers having different compositions, properties (e.g., acoustic transmission properties, viscosities, stiffnesses), configurations and dimensions may be provided and used with interface components 115 to provide a compatible interface for different types of probes, ultrasound controllers and systems, ultrasound protocols, and the like. In one embodiment, the acoustic coupler comprises a thermoplastic elastomer, such as an oil-enhanced or gelatinous thermoplastic elastomer. Suitable materials are described, for example, in U.S. Patent Publication 2005/0215901 A1. In the embodiment illustrated in FIGS. 9B, 10A and 10B, such a material forms an end face 116 of interface component 115 and, when probe housing 110 is mounted in interface component 115, acoustically emissive surface 111 of the inserted transducer intimately contacts the acoustic coupler forming end face 116 of interface component 115.

In the embodiments illustrated in FIGS. 10A and 10B, interface component 115 comprises a plurality of legs 117 extending from a rim-like structure 118 that supports the acoustic coupler 116 forming an end face of interface component 115. Legs 117 are sized and configured to be mounted (e.g., by sliding) over, and mate or interface with, an external surface of probe housing 110 and, in combination with rim-like structure 118, firmly secure interface component 115 on probe housing 110. Suitable mechanical and other types of interface structures are well known in the art. In the embodiment illustrated in FIGS. 10A and 10B, each interface component leg 117 incorporates a detent 119 that mates with a complementary notch 113 provided on probe housing 110. When interface component 115 is slidably mounted on probe housing 110, detents 119 lock into notches 113 to provide a stable and securely mated probe housing/interface assembly.

In some embodiments, interface component 115 and/or acoustic coupler 116 are intended for use in a single ultrasound operation and may be provided as single use and/or individual subject or ultrasound protocol accessories that are easily and conveniently mounted on a transducer housing and easily and conveniently removed from the transducer housing upon completion of an ultrasound protocol. In one embodiment, legs 117 of interface component 115 are designed and configured for stable, secure and convenient mounting and placement on probe housing 110, as described above, but cannot be removed from the probe housing without damaging or breaking the legs. In the embodiments illustrated in FIGS. 10A and 10B, for example, when interface component detents 119 are locked in probe housing notches 113 and the interface component is stably mounted on the probe housing, removal of the interface component may only be accomplished by bending or breaking one or more legs 117, thus discouraging, or preventing, use of the probe interface component 115 in multiple ultrasound protocols, or with multiple subjects.

In another embodiment, probe interface component 115 and/or acoustic coupler 116 may incorporate a coding component, such as an RFID identifier or another readable identifier that, when placed in proximity to probe housing 110, communicates with a complementary reading device to identify the interface component and/or acoustic coupler. In one embodiment, a confirming match or confirmation of an acceptable probe interface component may be required by the ultrasound system before the system is operable to conduct an ultrasound protocol. In another embodiment, different interface component(s) and/or acoustic coupler(s) may be required for operation with certain transducers or in certain ultrasound protocols. In one embodiment, a readable identifier required for system operation is associated with a component of the interface and/or acoustic coupler that, upon removal from the transducer, is non-functional to prevent re-use of the interface component and/or acoustic coupler.

When the probe assembly comprising probe housing 110 in combination with interface component 115 is installed in a mounted position in probe mount 90, see FIGS. 9A and 9B, acoustic coupler 116 is exposed through port 107 and positioned for contacting a subject's anatomical surface directly, or indirectly through another acoustically transmissive material or layer. The probe assembly is generally mountable in and removable from receiving portion 100 of probe mount 90 and is stably held in the probe mount during an ultrasound protocol. In one embodiment, interface component 115 is sized and configured to slidably mount, and lock, in receiving portion 100 during insertion and use and is releasable from receiving portion 100 following completion of an ultrasound protocol. In one embodiment, terminal portions 114 of interface component legs 117 mate with surfaces of the receiving portion 100 to lock the probe assembly in place within the probe mount during use, and allow for release under operator control following use.

The probe assembly when mounted in the receiving portion 100 of probe mount 90 may be adjusted, as described below, to align the acoustically emissive probe face(s) 111 and acoustic coupler 116 with desired target sites. The receiving portion 100 of probe mount 90 is illustrated in an exploded view in FIG. 9B. Probe mount receiving portion 100 preferably facilitates adjustment of a mounted probe assembly along angular and/or rotational paths and, in some embodiments, additionally provides adjustment of the probe assembly along a “z-axis,” toward and away from a target anatomical site on a subject.

In one embodiment, receiving portion 100 provides a gimbaled interface, or provides interaction of multiple partially spherical surfaces to provide adjustment of a mounted probe assembly along rotational paths with multiple degrees of freedom. In the embodiment illustrated in FIG. 9B, a probe assembly comprising an interface component 115 stably mounted on probe housing 110 is received in an interior cavity of curved cap section 101. The exterior curved surface of cap section 101 is retained in and adjustable with respect to curved surfaces formed by a combination of interior curved surfaces of components 102, 104 and 106, which are assembled on probe mount 90. When the probe assembly is mounted in receiving portion 100, it is adjustable with multiple degrees of freedom along a variety of angular paths, including rotational or partially spherical paths, by interaction of the exterior surface of cap section 101 along complementary curved surfaces of other receiving portion components. This adjustment feature provides a broad range of probe tilt and angular adjustment possibilities. A releasable locking mechanism that releasably fixes a desired rotational position of the probe assembly within receiving portion 100 is preferably provided.

In yet another embodiment, receiving portion 100 may be constructed and configured to provide adjustment of the probe assembly toward and away from stationary components of transducer mount 90 and framework structure 80. In one manifestation of this adjustment feature illustrated in FIG. 9B, receiving portion 100 comprises complementary rotational components 102, 104. Rotational component 102 comprises one or more projections 103 that mate with and ride in one or more slots 105 provided in mating rotational component 104. In this embodiment, a probe assembly comprising probe housing 110 in combination with interface component 115 mounted in the interior cavity of curved cap section 101 is retained along the interior curved wall of rotational component 102 and is adjustable by rotation of component 102 with respect to component 104, causing projections 103 to travel in slots 105 and moving the probe assembly along a z-axis, toward and away from the subject's anatomical surface. A releasable locking mechanism that releasably fixes a desired position of the probe assembly along the z-axis is preferably provided

In the embodiment illustrated in FIG. 9B, ultrasound probe 110 is movable along a lateral paths L and S, along rotational path R, along multiple angular, rotational and/or spherical paths by interaction of mating curved surfaces of the receiving portion 100, and along the z-axis, toward and away from a subject's anatomical surface. It will be appreciated that fewer than all of these adjustments may be provided in various other embodiments without departing from the scope of this invention. It will also be appreciated that additional adjustment features may be incorporated in alternative embodiments without departing from the scope of this invention.

In general, ultrasound probes are provided as reusable components and are used in combination with ultrasound diagnostic systems, such as TCD systems. Transducer interface components, such as 33, 76 and 115 are generally provided as single use components. In some embodiments, transducer interface components and probe mounts may be integrated and provided as reusable or single use components having specialized configurations for use with different types and configurations of ultrasound probes. Framework component(s) may be provided as reusable components, but may also be single use or patient specific components. In general, various components and features of the mounting systems described herein may be provided as modular components and features and combined, as necessary or desirable, to accommodate patient, diagnostic and monitoring requirements. Different configurations of transducer housings may be provided for interfacing with multiple configurations of transducers and transducer interface members, and various configurations of transducer housings may be mounted interchangeably on framework components having desired adjustment mechanisms. One having ordinary skill in the art will also appreciate that while the framework members for mounting to a subject's skull are shown, similar, differently configured systems having interchangeable components and various adjustment features may be provided for mounting to other body surfaces, e.g., neck, limbs, truck, and the like.

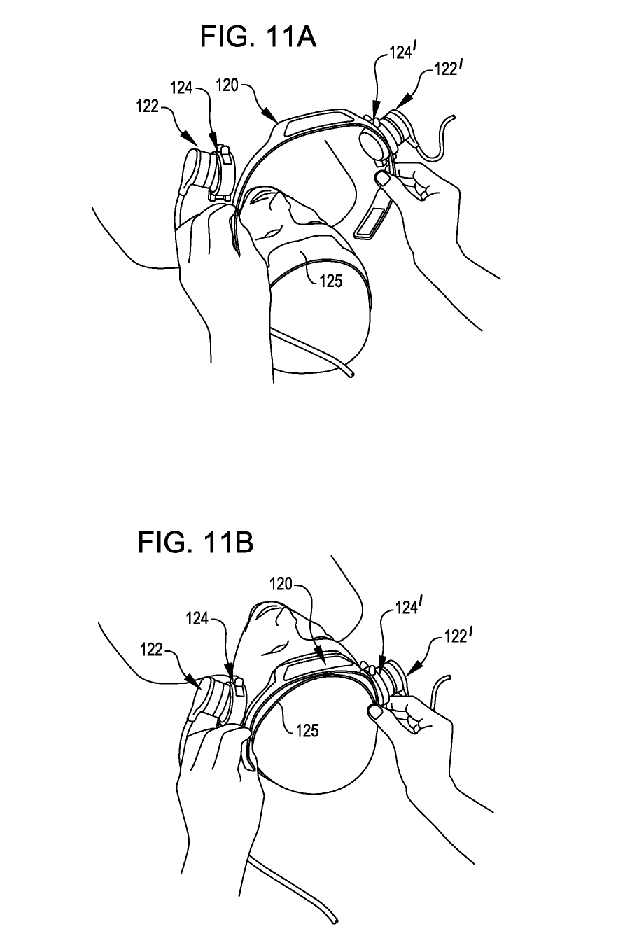

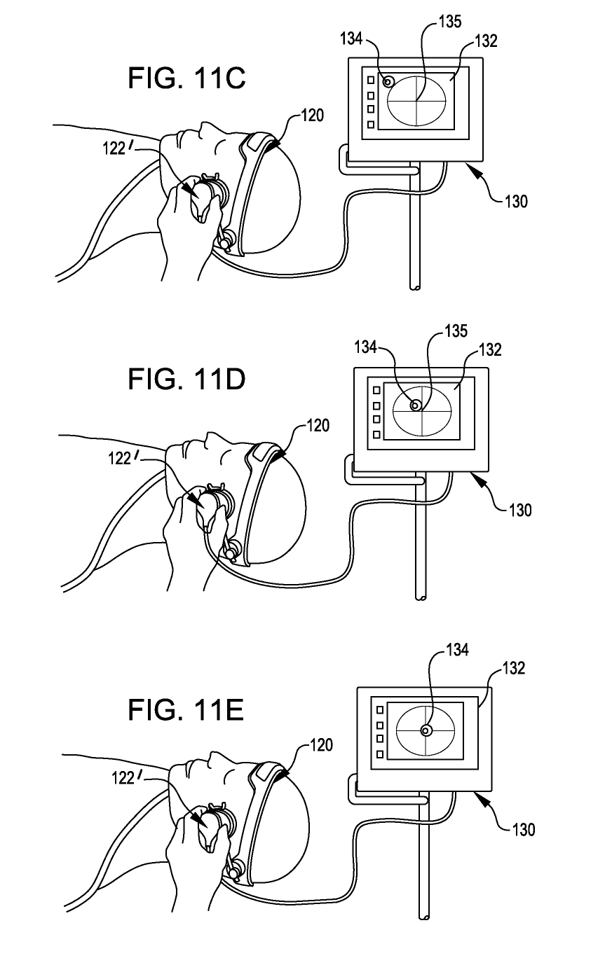

FIGS. 11A-11E illustrate the placement of a framework component with ultrasound probes mounted in probe housings on a subject's skull and adjustment of the position of the ultrasound transducer(s), using manual adjustment techniques in combination with a controller and display system, to aim the transducer(s) at a desired target site. Systems for automatically identifying a desired target site, locking ultrasound beam direction onto the target site, and periodically updating and reacquiring the target site are disclosed, for example, in U.S. Pat. Nos. 6,682,483; 7,399,279; 7,534,209; and 7,547,383, the disclosures of which are incorporated herein by reference in their entireties.

FIGS. 11A and 11B show the positioning of framework member 120 having ultrasound probes mounted in interface members 122, 122′, and interface members mounted adjustably in probe mounts 124, 124′ on a subject's skull. The framework member overlies headband 125 and probe mounts 124, 124′ are adjusted to position the acoustically emissive face of each of the probes at one of the subject's temporal windows, or at another desired location. FIG. 11C illustrates one of the probes operably connected to an ultrasound monitoring system 130 having a display 132. The object 134 shown on display 132 indicates the spatial location, in the patient's skull, of the ultrasound beams for a given orientation of the probe mounted in housing 124. A desired ultrasound examination target site may be input to the monitoring system by an operator, or a desired target site may be determined based upon feedback from the system during an ultrasound examination or protocol. In the embodiment illustrated in FIGS. 11C-11E, a desired target site 135 is indicated at the center of the cross displayed on the monitor, and the operator adjusts the position of the transducer, as shown schematically, until the ultrasound beam location matches, and overlies, the target site 135 indicated on the display. The ultrasound probe mount (and probe) may then be locked in place to maintain the ultrasound beam direction aligned with the desired target site. Target monitoring and re-acquisition protocols may be run periodically to maintain focus on, or re-acquire the target site.

We claim:

1. A frame member adapted to be mounted on a desired anatomical surface of a subject comprising two framework legs positioned opposite one another and a connecting member positioned to provide a bridge between the framework legs, at least one mounting structure extending adjustably from the frame member and at least one probe mount provided on the mounting structure.

2. A frame member of claim 1, wherein the at least one mounting structure is a mounting arm that is adjustable along an axial path and a pivotable path with respect to the frame member.

3. A frame member of claim 1, wherein the at least one probe mount has an interface surface for receiving a mating interface surface of an ultrasound probe, wherein the probe mount interface surface is curved and partially spherical and provides adjustment of the probe with respect to the probe mount interface surface with multiple degrees of freedom.

4. A frame member of claim 1, comprising at least one cut-out for receiving mounting elements constructed from a material different from the material of the frame member.

5. A frame member of claim 1, wherein the at least one probe mount is adjustable along at least three adjustment paths.

6. A frame member of claim 1, wherein the at least one probe mount is adjustable along at least one adjustment path in each of three dimensions.

7. In combination, a frame member of claim 1 and a flexible band adapted to be positioned on a patient's anatomical surface underneath the frame member.

8. The combination of claim 7, wherein the flexible band comprises at least one mounting structure configured to mate with a complementary mounting structure provided on the frame member.

9. A probe interface component adapted to provide an interface between an acoustic emission surface of an ultrasound probe and a subject's anatomical surface, the probe interface component comprising an acoustically transmissive interface portion providing a high fidelity acoustic coupler between the acoustic emission surface of the ultrasound probe and the subject's anatomical surface and a support structure adapted to be mounted on the ultrasound probe.

10. A probe interface component of claim 9 configured for stable mounting on the ultrasound probe, whereby removal of the probe interface component from the ultrasound probe disables the probe interface component and prevents re-use.

11. A probe interface component of claim 9, incorporating a coding component adapted for communication with a complementary reading device to identify the probe interface component.

12. In combination, a frame member of claim 1 and a probe interface component of claim 9.

13. An ultrasound probe mounting system comprising a receiving portion sized and configured for receiving an ultrasound probe and having a port for exposing an acoustically emissive face of the ultrasound probe, the receiving portion additionally comprising at least one curved surface adapted to interact with a complementary curved surface of an ultrasound probe assembly to provide tilting and angular adjustment of the ultrasound probe assembly within the receiving portion with multiple degrees of freedom.

14. The ultrasound probe mounting system of claim 13, wherein the ultrasound probe assembly and receiving portion are releasably lockable following positioning of the ultrasound probe assembly within the receiving portion.

15. An ultrasound probe mounting system comprising a receiving portion sized and configured for receiving an ultrasound probe and having a port for exposing an acoustically emissive face of the ultrasound probe, the receiving portion additionally providing adjustment of a mounted ultrasound probe along a z-axis.

16. The ultrasound probe mounting system of claim 15, wherein adjustment of the mounted ultrasound probe along a z-axis is provided by rotation of two mating components with respect to one another to move the components toward and away from one another.

17. An ultrasound monitoring system comprising an ultrasound probe in operable communication with an ultrasound controller for data acquisition, processing, analysis and/or display; a frame member having at least one mounting structure extending adjustably from the frame member and at least one ultrasound probe mount provided on the mounting structure, and a probe interface member adapted to provide an acoustic coupling interface between an acoustic emission surface of the ultrasound probe a subject's anatomical surface, the probe interface member comprising an acoustically transmissive interface portion and a mounting portion adapted to be mounted on the ultrasound probe.

18. A method for acquiring acoustic data from an ultrasound probe mountable in a framework structure positionable on a subject's anatomical surface, comprising: positioning the framework structure on a desired anatomical surface of the subject and, if necessary, adjusting the framework structure to provide stable positioning on the desired anatomical surface of the subject; positioning the ultrasound probe in a probe mount with an acoustically emissive face of the ultrasound probe exposed through a port of the probe mount and, if necessary, attaching the probe mount to the framework structure with the acoustically emissive face of the ultrasound probe in proximity to a desired ultrasound target; moving the probe mount along an axial path with respect to the framework structure to adjust the position of the acoustically emissive face of the ultrasound probe; moving the probe mount along a pivoting path with respect to the framework structure to adjust the position of the acoustically emissive face of the ultrasound probe; and locking the probe mount in a fixed position with respect to the framework structure when a desired position of the acoustically emissive face of the ultrasound probe is achieved.

19. The method of claim 18, additionally comprising tilting the acoustically emissive face of the ultrasound probe with respect to the probe mount and/or the framework structure to angularly adjust the position of the acoustically emissive face of the ultrasound probe prior to locking the probe mount in a fixed position.

20. The method of claim 19, wherein tilting the acoustically emissive face of the ultrasound probe is accomplished by moving complementary, partially spherical surfaces of the ultrasound probe housing and the probe mount with respect to one another.

21. The method of claim 19, wherein tilting the acoustically emissive face of the ultrasound probe is accomplished by moving complementary, partially spherical surfaces of the probe mount with respect to one another.

22. The method of claim 18, additionally comprising moving the acoustically emissive face of the ultrasound probe in a z-axis with respect to the probe mount and/or the framework structure to adjust the position of the acoustically emissive face of the ultrasound probe prior to locking the probe mount in a fixed position.

23. The method of claim 22, wherein moving the acoustically emissive face of the ultrasound probe in a z-axis is accomplished by rotating complementary components of the probe mount and thereby moving the complementary components of the probe mount toward and away from one another.

24. The method of claim 18, additionally comprising mounting a probe interface component having an acoustically transmissive coupling member on the ultrasound probe prior to positioning the ultrasound probe in the probe mount to provide a probe assembly having an acoustic coupling member for contacting the subject's anatomical surface, whereby the acoustically transmissive coupling member is positioned in proximity to the acoustically emissive face of the ultrasound probe.

25. The method of claim 24, additionally comprising removing the ultrasound probe assembly from the probe mount following acquisition of ultrasound data and separating the probe interface component from the ultrasound probe.

26. The method of claim 24, additionally comprising separating the probe interface component from the ultrasound probe by substantially interfering with the integrity of the probe interface component, thereby preventing re-use of the probe interface component.
